(19) United States
(12) Patent Application Publication
(10) Pub. No.: US 2026/0309320 A1
(43) Pub. Date: Oct. 8, 2026

(54) LUMINAIRE PROVIDED WITH A UV LIGHTING MODULE

(71) Applicant: SIGNIFY HOLDING B.V., EINDHOVEN (NL)

(72) Inventors: TIES VAN BOMMEL, HORST (NL); RÉMY CYRILLE BROERSMA, EINDHOVEN (NL)

(21) Appl. No.: 19/481,000

(22) PCT Filed: Apr. 22, 2024
(86) PCT No.: PCT/EP2024/060894

§ 371 Date: Nov. 3, 2025

(30) Foreign Application Priority Data
May 2, 2023 (EP) 23170979.1

(51) Int. Cl. F21V 33/00; F21S 8/04; F21V 23/04; F21V 23/06; F21Y 113/00; F21Y 113/20

(52) U.S. Cl. CPC F21V 33/0064; F21S 8/04; F21V 23/0471; F21V 23/06; F21Y 2113/20; F21Y 2113/30

(57) ABSTRACT

A luminaire system comprising a luminaire (1) having a connection slot (6) and a UV lighting module (7) comprising a connection portion (11) configured to be inserted into the connection slot (6), a light emitting portion (12) housing a module solid state light source (13) configured to generate module light (32) comprising UV light having a dominant peak wavelength in the 100-380 nm range, and a light exit window (14) configured to exit the module light, wherein an area (A2) of the light exit window (14) is at least 1.5 times as large as an opening area (Al) of the connection portion (11). With this design, a relatively small connection slot may be used to connect a UV lighting module with a significantly larger light exit window. This makes it possible to easily retrofit a luminaire provided with a “sensor slot” with a UV lighting capability.

### FIELD OF THE INVENTION

The present invention relates to UV illumination, and particularly to a luminaire having a releasably attached UV lighting module.

### BACKGROUND OF THE INVENTION

UVB light has properties beneficial for humans, such as promoting the production of vitamin B. UVB light is a component of natural sunlight, but in modern society many people are confined to spending much of their time indoors. Artificial UVB light can then be advantageous to maintain a healthy vitamin D production.

Artificial UVB light may be provided in many ways and in many contexts. However, it would be beneficial to provide artificial UVB light as an integrated part of general illumination, e.g. in an office space. In particular, it would be beneficial if existing luminaires could be easily retro-fitted to also provide UVB lighting.

In EP 3660385 a lighting device is disclosed that includes at least one first light source emitting a visible light, at least one second light source disposed to be spaced apart from the first light source and emitting a light having a sterilization wavelength, and a housing at which the first light source and the second light source are disposed. The first light source is disposed outside an area having an angle corresponding to about 50 percent of a maximum amount of the light emitted from the second light source based on a line substantially perpendicular to a center of the second light source.

### SUMMARY OF THE INVENTION

It is an object of the present invention to provide a UV lighting module which may be retrofitted in existing luminaires.

According to a first aspect of the invention, this and other objects are achieved by a luminaire system comprising a luminaire including a luminaire light source configured to emit white light and a connection slot and a UV lighting module. The UV lighting module comprises a connection portion configured to be inserted into the connection slot so as to be releasably attachable, a light emitting portion housing a module solid state light source configured to generate module light comprising UV light having a dominant peak wavelength in the 280-320 nm range, and a light exit window configured to exit the module light. A window area of the light exit window is at least 1.5 times as large as an opening area of the connection portion.

White light is here intended broadly but may for example be light having a correlated colour temperature in the range 1500K-6500K, and e.g. a CRI of at least 80.

With this system, a connection slot such as one intended for connection of sensor modules, is used to connect the UV lighting module to a white light luminaire.

Connection slots for connecting various sensors to a luminaire are known e.g. the “upgradeable sensor slot” from Philips. The conventional understanding, and common practice, is that any module connected to such a slot has essentially the same dimensions as the slot. Such existing slots have therefore not been considered for UV-lighting, as a larger exit surface is required to avoid a too high intensity of UV light. The present invention is based on the realization that a UV lighting module can be designed to have a connection portion configured to fit in the connection slot, and a light exit surface which is larger than the slot opening area. By “opening area” is intended a cross section of an opening of the slot in which the connection portion is received.

With this design, a relatively small connection slot (e.g. a few cm wide) may be used to connect a UV lighting module with a significantly larger light exit window). This makes it possible to easily retrofit a luminaire provided with a “sensor slot” with a UV lighting capability.

It is noted that the present invention is equally applicable to module light sources emitting other UV wavelengths than UVB light (i.e. 280-320 nm) such as UVA (320-380 nm) and/or UVC (100-280 nm) light.

Preferably, the area of the light exit window is twice as large as the slot cross section. As an example, the light exit window may have a width in the range 10-60 cm, which is considerably larger than known connection slots.

In some embodiments, the UV lighting module has a general shape of a T, wherein the connection portion forms the base of the T, and the UV light emitting portion forms the bar of the T, such that, when the UV lighting module is connected in the slot, the light exit window extends on two opposite sides over the slot. This design is beneficial as it allows the larger size of the light exit window extend only in two directions, thereby making it easier to avoid obstructing light from a luminaire in which the module is mounted.

In some embodiment, the UV light emitting portion further comprises an optical element configured to diffuse or scatter the module light. This may be beneficial, especially if UV radiation is intended to illuminate a particular object, or if it is intended to be homogeneously spread in the ambient.

In some embodiments, the UV light emitting portion comprises a further module solid state light source configured to generate further module light comprising violet light having a (dominant) peak wavelength in the 380-420 nm range.

The module may comprise a controller for individually controlling the luminaire light source and the module light source. The controller may be configured for individually controlling white light emission (e.g. in terms of intensity, color point, correlated color temperature and/or color rendering index) from the luminaire light source and/or module light emission (e.g. intensity) from the module solid state light source. Such a controller may further be functionally connected to a (remotely arranged) indicator (e.g. visual, light, etc.) and be configured to activate the indicator when the module light source is activated.

The module may further comprise a comprise a (proximity) sensor functionally connected to the controller and configured to provide a (proximity) sensor signal e.g. indicating presence of an object (such as a person or animal) in a field of view of the proximity sensor, wherein the controller is configured to control the module solid state light source (and/or the white light emission from the luminaire light source) in dependence on the proximity sensor signal. For example, the controller may be configured to dim, or even deactivate, the module solid state light source when there is no one in the field of view. Alternatively, other sensors may be used such as for example IR-detectors, radar range detectors, sound detectors, etc.

The connection slot is preferably configured to provide a mechanical connection for securing the UV lighting module in the luminaire, and an electrical connection for providing electrical power to the UV lighting module. As an example, the connection slot may have a width in the range 1-8 cm.

In some embodiments, the luminaire includes a central light emitting portion and a peripheral rim at least partly surrounding the central portion, and the connection slot is provided in this rim. This allows mounting a UV lighting module without obstructing the light from the luminaire. Preferably, the light exit window of the UV lighting device is elongated and has a longitudinal axis aligned with said rim. This ensures that the UV light emitting portion including the light exit window does not extend over the light emitting portion of the luminaire.

### BRIEF DESCRIPTION OF THE DRAWINGS

This and other aspects of the present invention will now be described in more detail, with reference to the appended drawings showing embodiments of the present invention.

FIG. 1 is a perspective view of a luminaire provided with a connection slot for receiving a UV module according to an embodiment of the invention.

FIG. 2 shows the UV module in more detail.

FIG. 3 shows a room provided with two luminaires as shown in FIG. 1.

### DETAILED DESCRIPTION

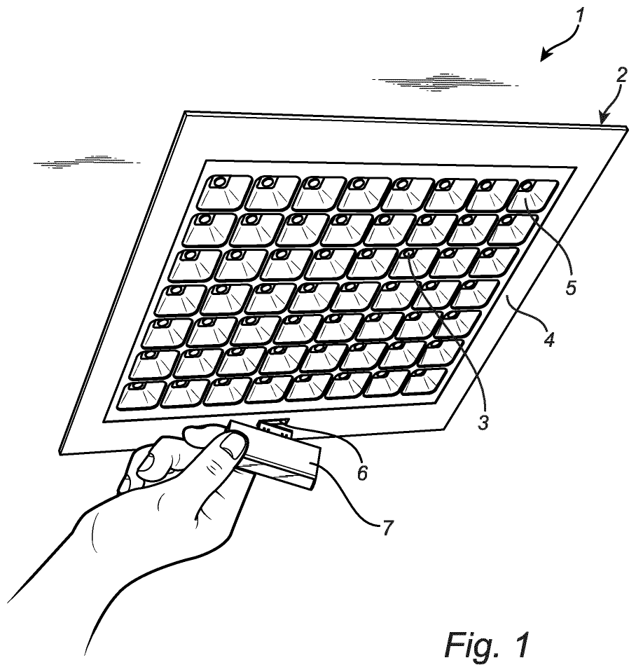

The luminaire 1 in FIG. 1 includes a housing 2 and a plurality of light sources 3 arranged in the housing 2. The light sources 3 are typically solid state light sources such as LED light sources.

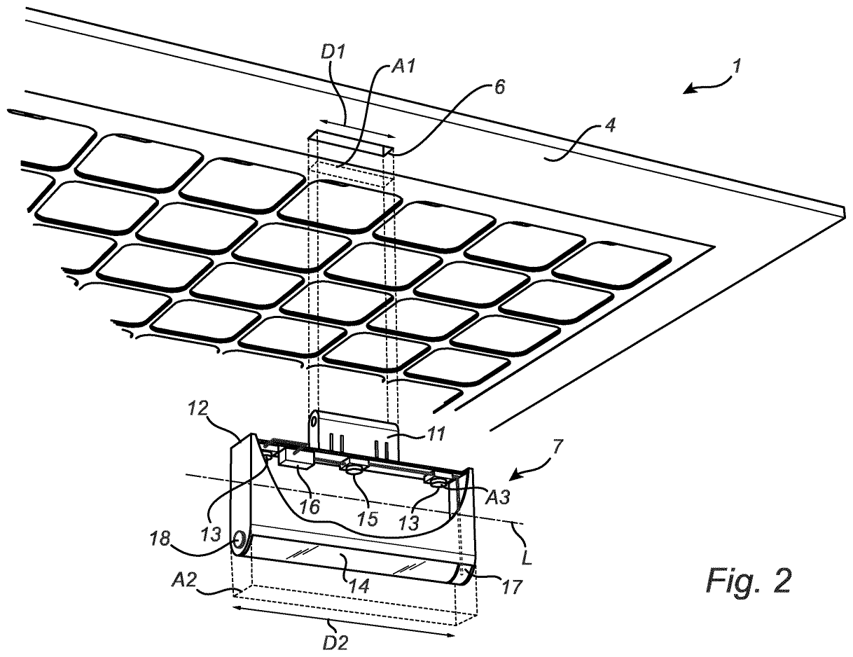

As shown in FIG. 1, the housing 2 forms a frame 4 surrounding a light generation portion 5 where the light sources 3 are arranged. The light sources 3 are typically configured to emit white, office-compliant light. In the frame 4 is provided a connection slot 6, configured to receive a plug-in module 7 such that the module 7 is releasably attachable to the luminaire 1. Various types of modules can be connected, including e.g. various sensor modules. Various luminaires with this type of “sensor slot” are sold by Philips®. Each connection slot 6 has an opening to receive the plug-in module 7, and this opening has a width D1 (i.e. extension in the longest dimension) and an opening area A1 (see FIG. 2). As an example, the width may be a few centimeters, e.g. 1-8 cm, preferably 3-5 cm. The area A1 may be in the range 2-20 cm².

Releasable mechanical connection of module 7 in the sensor slot 6 may be provided in a snap-in manner, e.g. by means of a clip or bracket. Electrical connection of the module 7 may be provided by a galvanic plug-and-socket type connection, or by a wireless connection, e.g. through induction. Alternatively, the module 7 may be provided with an on-board power supply, such as a disposable or rechargeable battery (not shown). Details of suitable mechanical and electrical connections are well-known in the art, and will not be discussed in detail herein.

According to aspects of the present invention, the module 7 is a UV lighting module configured to emit UV light. This UV module 7 will be described in more detail with reference to FIG. 2.

The UV module 7 has a connection portion 11, configured to be inserted into the connection slot 6 so as to be mechanically secured therein. The UV module 6 further has a UV light emitting portion 12, housing at least one, in the illustrated case two, solid state light source 13 configured to generate UV light (referred to as module light) having a (dominant) peak wavelength in the 280-320 nm range. The light emitting portion 12 has a light exit window 14 configured to exit the UV light into the ambient. The light emitting portion 12 may operate as a light mixing chamber, in which case the exit window 14 is a transparent cover, e.g. plastic or glass. Alternatively, the exit window 14 is simply an opening.

In some embodiments, the light emitting portion 12 further houses at least one solid state light source 15 configured to generate further module light comprising violet light having a (dominant) peak wavelength in the 380-420 nm range.

The window area A2 of the light exit window 14 is at least 1.5 times as large as the opening area Al of the connection slot 6. As an example, the light exit window can have a width D2 (i.e. extension in the longest dimension) in the range 10-60 cm, preferably 15-25 cm. In other words, several times longer than the connection slot 6. The area A2 may be in the range 10-100 cm². This means that relatively high intensity UV light emitted from the LED(s) 13 may be distributed in the light emitting portion 12, before it is transmitted though the light exit window 14 with less intensity. The LED(s) 13 each have a light output surface area A3 which may be less than ⅕ of the opening area A1. If there are several LEDs 13, then the total light surface area (i.e. the sum of each area A3) may be less than ⅕ of the opening area A1.

In the illustrated embodiment, the UV module 7 has the shape of a T, where the connection portion 11 forms the base of the T and the light emitting portion 12 forms the bar of the T. As shown, when the UV module 7 is inserted into the slot 6, distal portions 12a, 12b of the light emitting portion 12, and consequently the light exit window 14, extend on opposite sides of the slot 6. Further, when the module 7, like in the illustrated case, is aligned with the frame 4, the light exit window 14 will not extend in front of the light emitting portion 5 of the luminaire 1.

The light exit window 14 may be adapted to, or may comprise a separate optical element configured to, diffuse or scatter the light from the LEDs. The light exit window may be curved, to provide an improved distribution of emitted light.

A controller 16 is connected to control the LEDs 13, 15.

In the illustrated example, the LED module 6 further comprises a proximity sensor 17, e.g. an IR sensor or UWB radar sensor. The sensor can be configured to detect when an object is in a field of view of the sensor 17. Alternatively, the sensor can be configured to detect movement within the field of view. The sensor 17 may also be connected to the controller 16, to thereby provide proximity-based control of the emitted light. For example, the controller may be configured to only activate the LEDs 13, 15 in a situation where a living being is determined to be in a field of view of the sensor 17. This may avoid UV emission when there is no-one in a room.

The module 7 may also comprise an indicator 18, e.g. a visible light indicator, connected to the controller 16. The controller can be configured to activate the indicator 18 whenever the UV light source 13 is activated, thereby making a person in the vicinity aware of the UV light emission.

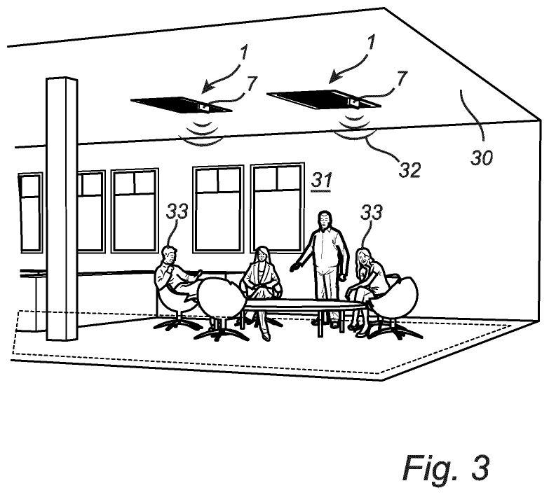

FIG. 3 shows two luminaires 1 provided with UV modules 7 suspended in a ceiling 30 of a room 31. Light 32 emitted from the UV modules 7 is incident on two people 33 present in the room 31, thereby ensuring a healthy vitamin D production in these people.

The person skilled in the art realizes that the present invention by no means is limited to the preferred embodiments described above. On the contrary, many modifications and variations are possible within the scope of the appended claims. For example, other types of luminaires, provided with appropriate connection slots, can be combined with a UV module according to the invention.

1. A luminaire system comprising:
a luminaire including at least one luminaire light source configured to emit white light and a connection slot; and
a UV lighting module, emitting at least one of UVA, UVB or UVC light, comprising:
a connection portion, configured to be inserted into said connection slot so as to be releasably attachable;
a light emitting portion housing a module solid state light source configured to generate module light comprising UV light having a dominant peak wavelength in the 100-380 nm range, and a light exit window configured to exit said module light,
wherein a window area of said light exit window is at least 1.5 times as large as an opening area of the connection portion, and
wherein the UV lighting module has a general shape of a T, wherein the connection portion forms a base of the T, and the UV light emitting portion forms a bar of the T, such that, when the UV lighting module is connected in a connection slot, the light exit window extends on two opposite sides of the connection slot.

2. The luminaire system according to claim 1, wherein said module solid state light source has a light output surface area being less than ⅕ of said opening area.

3. The luminaire system according to claim 1, wherein the UV light exit window further comprises an optical element configured to diffuse or scatter the module light.

4. The luminaire system according to claim 1, wherein the light exit window has a width in the range 10-60 cm.

5. The luminaire system according to claim 1, wherein said UV light emitting portion further comprises a further module solid state light source configured to generate further module light comprising violet light having a dominant peak wavelength in the 380-420 nm range.

6. The luminaire system according to claim 1, further comprising a controller for individually controlling white light emission from the luminaire light source and module light emission from the module solid state light source.

7. The luminaire system according to claim 6, further comprising an indicator functionally connected to said controller, and wherein said controller is configured to activate said indicator when said module solid state light source is activated.

8. The luminaire system according to claim 6, further comprising a proximity sensor functionally connected to the controller and configured to provide a proximity sensor signal indicating presence of an object in a field of view of the proximity sensor, wherein the controller is configured to control the module solid state light source in dependence on the proximity sensor signal.

9. The luminaire system according to claim 1, wherein the connection slot is configured to provide a mechanical connection for releasably securing said UV lighting module in the luminaire.

10. The luminaire system according to claim 1, wherein the connection slot is configured to provide electrical power to the UV lighting module.

11. The luminaire system according to claim 1, wherein the luminaire includes a central light generation portion and a peripheral rim at least partly surrounding the central light generation portion, and wherein the connection slot is provided in said rim.

12. The luminaire system according to claim 11, wherein the light exit window of the UV lighting module is elongated with a longitudinal axis aligned with said rim, so that the UV light module does not overlap the central light generation portion.

13. The luminaire system according to claim 1, wherein the connection slot has a width in the range 1-8 cm.
